(19) United States
(12) Patent Application Publication
(10) Pub. No.: US 2021/0007575 A1
(43) Pub. Date: Jan. 14, 2021

(54) IMAGE PROCESSING DEVICE, ENDOSCOPE SYSTEM, IMAGE PROCESSING METHOD, AND COMPUTER-READABLE RECORDING MEDIUM

(71) Applicant: OLYMPUS CORPORATION, Tokyo (JP)

(72) Inventor: Sunao KIKUCHI, Tokyo (JP)

(73) Assignee: OLYMPUS CORPORATION, Tokyo (JP)

(21) Appl. No.: 17/012,149

(22) Filed: Sep. 4, 2020

Related U.S. Application Data
(63) Continuation of application No. PCT/JP2018/009816, filed on Mar. 13, 2018.

(51) Int. Cl. A61B 1/00; G06T 7/00; A61B 1/06

(52) U.S. Cl. CPC A61B 1/00009; G06T 7/0012; G06T 2207/10068; G06T 2207/10024; A61B 1/0638

(57) ABSTRACT

Provided is an image processing device including a processor including hardware, the processor being configured to detect a positional deviation amount of pixels among image data of a plurality of frames; combine, based on the detected positional deviation amount, information concerning the pixels, in which a first filter is arranged, of the image data of at least one or more past frames with image data of a reference frame to generate combined image data; perform interpolation processing on the generated combined image data to generate, as reference image data, first interpolated image data including information concerning the first filter in all pixel positions; and perform, referring to the generated reference image data, interpolation processing on the image data of the reference frame to generate, for each of a plurality of types of second filters, second interpolated image data including information concerning the second filters in all pixel positions.

### CROSS-REFERENCE TO RELATED APPLICATION

This application is a continuation of International Application No. PCT/JP2018/009816, filed on Mar. 13, 2018, the entire contents of which are incorporated herein by reference.

### BACKGROUND

### 1. Technical Field

The present disclosure relates to an image processing device for performing image processing on an imaging signal captured by an endoscope, an endoscope system, and an image processing method, and a computer-readable recording medium.

### 2. Related Art

In the medical field and the industrial field, endoscope apparatuses have been widely used for various tests. Among the endoscope apparatuses, an endoscope apparatus for medical use can acquire, by inserting an elongated flexible insertion unit, at the distal end of which an imaging element including a plurality of pixels is provided, into a body cavity of a subject such as a patient, an in-vivo image in the body cavity without dissecting the subject. Therefore, load on the subject is small. The endoscope apparatus have been spread.

As an imaging scheme of the endoscope apparatus, sequential lighting for irradiating illumination in a different wavelength band for each of frames to acquire color information and simultaneous lighting for acquiring color information with a color filter provided on an imaging element are used. The sequential lighting is excellent in color separation performance and resolution. However, color shift occurs in a dynamic scene. On the other hand, in the simultaneous lighting, color shift does not occur. However, the simultaneous lighting is inferior to the sequential lighting scheme in color separation performance and resolution.

As an observation scheme of an endoscope apparatus in the past, white light imaging (WLI) using white illumination light (white light) and a narrow band imaging (NBI) using illumination light (narrow band light) including two narrow band lights respectively included in wavelength bands of blue and green are well known. In the white light imaging, a color image is generated using a signal in the wavelength band of green as a luminance signal. In the narrow band imaging, a pseudo color image is generated using a signal in the wavelength band of blue as a luminance signal. Of the white light imaging and the narrow band imaging, the narrow band imaging can obtain an image for highlighting capillaries, mucosa micro patterns, and the like present in a mucosa surface layer of an organism. With the narrow band imaging, it is possible to more accurately find a lesioned part in the mucosa surface layer of the organism. Concerning such an observation scheme of the endoscope apparatus, it is also known that the white light imaging and the narrow band imaging are switched to perform observation.

In order to generate and display a color image with the observation scheme explained above, a color filter generally called Bayer array is provided on a light receiving surface of the imaging element to acquire a captured image with a single-plate imaging element. In this case, pixels receive light in a wavelength band transmitted through the filter and generate electric signals of color components corresponding to the light in the wavelength band. Accordingly, in processing for generating a color image, interpolation processing for interpolating signal values of color components lacked without being transmitted thorough the filter in the pixels is performed. Such interpolation processing is called demosaicing processing. A color filter generally called Bayer array is provided on the light receiving surface of the imaging element. In the Bayer array, filters that transmit lights in wavelength bands of red (R), green (G), and blue (B) (hereinafter referred to as “filter R”, “filter G”, and “filter B”) are arrayed for each of pixels as one filter unit.

In recent years, there has been known a technique of filter arrangement in which not only primary color filters but also complementary color filters of complementary colors such as cyan (Cy) or magenta (Mg) (hereinafter referred to as “filter Cy” and “filter Mg”) are mixed in order to obtain high resolution feeling in both of the white light imaging and the narrow band imaging in an organism (JP 2015-116328 A). With this technique, by mixing complementary color pixels, more information in a blue wavelength band can be acquired compared with the case of only primary color pixels. Therefore, it is possible to improve resolution of capillaries and the like in the case of the narrow band imaging.

### SUMMARY

In some embodiments, provided is an image processing device including a processor comprising hardware, the image processing device to which an endoscope is connectable, the endoscope including an image sensor and a color filter, the image sensor including a plurality of pixels arranged in a two-dimensional lattice shape, each pixel being configured to receive and photoelectrically convert lights to generate image data in a predetermined frame, the color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the pixels, the processor being configured to: detect a positional deviation amount of the pixels among the image data of a plurality of frames generated by the image sensor; combine, based on the detected positional deviation amount, information concerning the pixels, in which the first filter is arranged, of the image data of at least one or more past frames with image data of a reference frame to generate combined image data; perform interpolation processing on the generated combined image data to generate, as reference image data, first interpolated image data including information concerning the first filter in all pixel positions; and perform, referring to the generated reference image data, interpolation processing on the image data of the reference frame to generate, for each of the plurality of types of second filters, second interpolated image data including information concerning the second filters in all pixel positions.

In some embodiments, provided is an endoscope system including: an endoscope configured to be inserted into a subject; and an image processing device to which the endoscope is connected. The endoscope includes: an image sensor in which a plurality of pixels arranged in a two-dimensional lattice shape, each pixel being configured to receive and photoelectrically convert lights to generate image data in a predetermined frame; and a color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the pixels. The image processing device includes a processor comprising hardware, the processor being configured to: detect a positional deviation amount of the pixels among the image data of a plurality of frames generated by the image sensor; combine, based on the detected positional deviation amount, information concerning the pixels, in which the first filter is arranged, of the image data of at least one or more past frames with image data of a reference frame to generate combined image data; perform interpolation processing on the generated combined image data to generate, as reference image data, first interpolated image data including information concerning the first filter in all pixel positions; and perform, referring to the generated reference image data, interpolation processing on the image data of the reference frame to generate, for each of the plurality of types of second filters, second interpolated image data including information concerning the second filters in all pixel positions.

In some embodiments, provided is an image processing method executed by an image processing device to which an endoscope is connectable, the endoscope including an image sensor and a color filter, the image sensor including a plurality of pixels arranged in a two-dimensional lattice shape, each pixel being configured to receive and photoelectrically convert lights to generate image data in a predetermined frame, the color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the pixels. The image processing method includes: detecting a positional deviation amount of the pixels among the image data of a plurality of frames generated by the image sensor; combining, based on the detected positional deviation amount, information concerning the pixels, in which the first filter is arranged, of the image data of at least one or more past frames with image data of a reference frame to generate combined image data; performing interpolation processing on the generated combined image data to generate, as reference image data, first interpolated image data including information concerning the first filter in all pixel positions; and performing, referring to the generated reference image data, interpolation processing on the image data of the reference frame to generate, for each of the plurality of types of second filters, second interpolated image data including information concerning the second filters in all pixel positions.

In some embodiments, provided is a non-transitory computer-readable recording medium with an executable program stored thereon. The program causes an image processing device to which an endoscope is connectable, the endoscope including image sensor and a color filter, the image sensor including a plurality of pixels arranged in a two-dimensional lattice shape, each pixel being configured to receive and photoelectrically convert lights to generate image data in a predetermined frame, the color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the plurality of pixels, to execute: detecting a positional deviation amount of the pixels among the image data of a plurality of frames generated by the image sensor; combining, based on the detected positional deviation amount, information concerning the pixels, in which the first filter is arranged, of the image data of at least one or more past frames with image data of a reference frame to generate combined image data; performing interpolation processing on the generated combined image data to generate, as reference image data, first interpolated image data including information concerning the first filter in all pixel positions; and performing, referring to the generated reference image data, interpolation processing on the image data of the reference frame to generate, for each of the plurality of types of second filters, second interpolated image data including information concerning the second filters in all pixel positions.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic configuration diagram of an endoscope system according to a first embodiment of the present disclosure;

FIG. 2 is a block diagram illustrating a functional configuration of the endoscope system according to the first embodiment of the present disclosure;

FIG. 3 is a schematic diagram illustrating an example of a configuration of a color filter according to the first embodiment of the present disclosure;

FIG. 4 is a diagram illustrating an example of transmission characteristics of filters configuring the color filter according to the first embodiment of the present disclosure;

FIG. 5 is a diagram illustrating an example of spectral characteristics of lights emitted by a light source according to the first embodiment of the present disclosure;

FIG. 6 is a diagram illustrating an example of a spectral characteristic of narrowband light emitted by a light source device according to the first embodiment of the present disclosure;

FIG. 7 is a flowchart illustrating an overview of processing executed by a processor device according to the first embodiment of the present disclosure;

FIG. 8 is a diagram schematically illustrating an image generated by the processor device according to the first embodiment of the present disclosure;

FIG. 9 is a schematic diagram illustrating an example of a configuration of a color filter according to a second embodiment of the present disclosure;

FIG. 10 is a schematic diagram illustrating an example of transmission characteristics of filters configuring the color filter according to the second embodiment of the present disclosure;

FIG. 11 is a flowchart illustrating an overview of processing executed by the processor device according to the first embodiment of the present disclosure;

FIG. 12 is a diagram schematically illustrating an image generated by the processor device according to the first embodiment of the present disclosure;

FIG. 13 is a block diagram illustrating a functional configuration of an image processing unit according to a third embodiment of the present disclosure;

FIG. 14 is a flowchart illustrating an overview of processing executed by the processor device according to the first embodiment of the present disclosure;

FIG. 15 is a diagram schematically illustrating an image generated by the processor device according to the first embodiment of the present disclosure;

FIG. 16 is a flowchart illustrating an overview of processing executed by the processor device according to the first embodiment of the present disclosure;

FIG. 17 is a diagram schematically illustrating an image generated by the processor device according to the first embodiment of the present disclosure; and

FIG. 18 is a schematic diagram illustrating an example of a configuration of a color filter according to a modification of first to fourth embodiments of the present disclosure.

### DETAILED DESCRIPTION

Modes for carrying out the present disclosure (hereinafter referred to as “embodiments”) are explained below. In the embodiments, an endoscope apparatus for medical use that captures an image of the inside of a body cavity of a subject such as a patient and displays the image is explained. The disclosure is not limited by the embodiments. Further, in the description of the drawings, the same portions are denoted by the same reference numerals and signs and explained.

### First Embodiment

Configuration of an Endoscope System

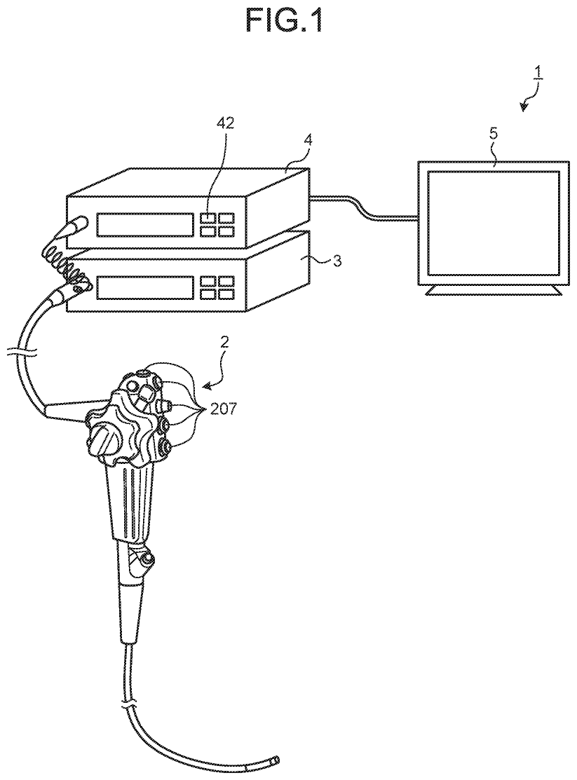

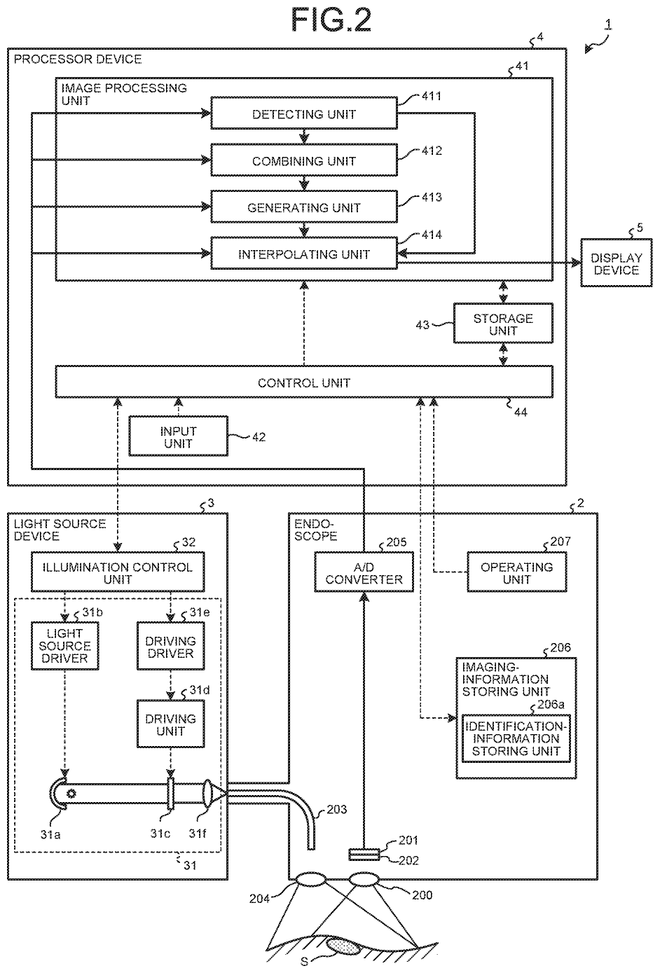

FIG. 1 is a schematic configuration diagram of an endoscope system according to a first embodiment of the present disclosure. FIG. 2 is a block diagram illustrating a functional configuration of the endoscope system according to the first embodiment of the present disclosure.

An endoscope system 1 illustrated in FIG. 1 and FIG. 2 is inserted into a subject such as a patient and images the inside of a body of the subject and outputs an in-vivo image corresponding to image data of the inside of the body to an external display device. A user such as a doctor observes the in-vivo image displayed by the display device to thereby test presence or absence of a bleeding site, a tumor site, and an abnormal site, which are detection target sites.

The endoscope system 1 includes an endoscope 2, a light source device 3, a processor device 4, and a display device 5. The endoscope 2 is inserted into the subject to thereby image an observed region of the subject and generate image data. The light source device 3 supplies illumination light emitted from the distal end of the endoscope 2. The processor device 4 applies predetermined image processing to the image data generated by the endoscope 2 and collectively controls the operation of the entire endoscope system 1. The display device 5 displays an image corresponding to the image data to which the processor device 4 has applied the image processing.

Configuration of the Endoscope

First, a detailed configuration of the endoscope 2 is explained.

The endoscope 2 includes an imaging optical system 200, an imaging element 201, a color filter 202, a light guide 203, a lens for illumination 204, an A/D converter 205, an imaging-information storing unit 206, and an operating unit 207.

The imaging optical system 200 condenses at least light from the observed region. The imaging optical system 200 is configured using one or a plurality of lenses. Note that an optical zoom mechanism for changing an angle of view and a focus mechanism for changing a focus may be provided in the imaging optical system 200.

The imaging element 201 is formed by arranging, in a two-dimensional matrix shape, pixels (photodiodes) that receive lights. The imaging element 201 performs photoelectric conversion on the lights received by the pixels to thereby generate image data. The imaging element 201 is realized using an image sensor such as a CMO (Complementary Metal Oxide Semiconductor) or a CCD (Charge Coupled Device).

The color filter 202 includes a plurality of filters arranged on light receiving surfaces of the pixels of the imaging element 201, each of the plurality of filters transmitting light in an individually set wavelength band.

Configuration of the Color Filter

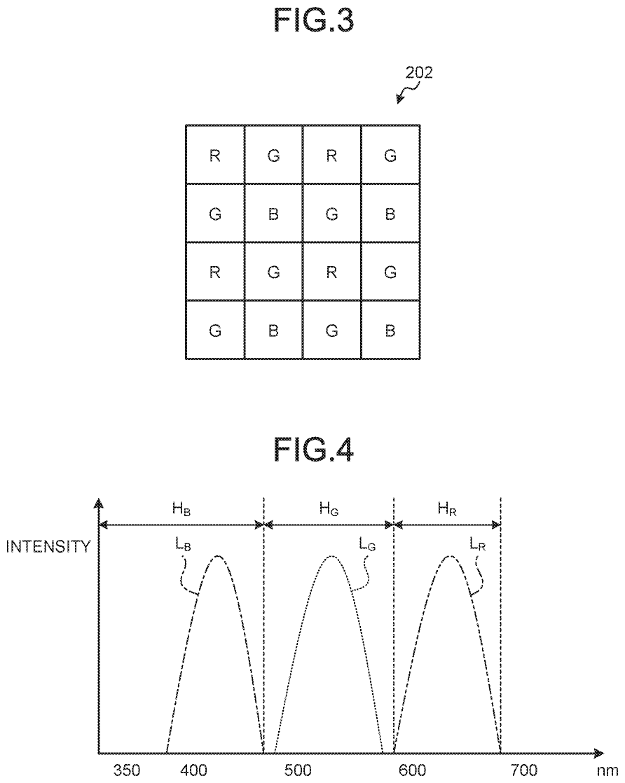

FIG. 3 is a schematic diagram illustrating an example of a configuration of the color filter 202. The color filter 202 illustrated in FIG. 3 is formed in a Bayer array configured by an R filter that transmits light in a wavelength band of red, two G filters that transmit light in a wavelength band of green, and a B filter that transmits light in a wavelength band of blue. A pixel P, in which the R filter that transmits the light in the wavelength band of red is provided, receives the light in the wavelength band of red. The pixel P that receives the light in the wavelength band of red is hereinafter referred to as R pixel. Similarly, the pixel P that receives the light in the wavelength band of green is referred to as G pixel, and the pixel P that receives the light in the wavelength band of blue is referred to as B pixel. Note that, in the following explanation, the R pixel, the G pixel, and the B pixel are explained as primary color pixels. As wavelength bands H_{B}, H_{G}, and H_{R} of blue, green, and red, the wavelength band H_{B} is 390 nm to 500 nm, the wavelength band H_{G} is 500 nm to 600 nm, and the wavelength band H_{R} is 600 nm to 700 nm.

Transmission Characteristics of the Filters

FIG. 4 is a diagram illustrating an example of transmission characteristics of the filters configuring the color filter 202. Note that, in FIG. 4, transmittance curves are simulatively standardized such that maximum values of transmittances of the filters are equal. In FIG. 4, a curve L_{B} indicates a transmittance curve of the B filter, a curve L_{G} indicates a transmittance curve of the G filter, and a curve L_{R} indicates a transmittance curve of the R filter. In FIG. 4, the horizontal axis indicates a wavelength (nm) and the vertical axis indicates transmittance (sensitivity).

As illustrated in FIG. 4, the B filter transmits light in the wavelength band H_{B}. The G filter transmits light in the wavelength band H_{G}. The R filter transmits light in the wavelength band H_{R}. In this way, the imaging element 201 receives lights in the wavelength bands corresponding to the filters of the color filter 202.

Referring back to FIG. 1 and FIG. 2, the explanation of the configuration of the endoscope system 1 is continued.

The light guide 203 is configured using a glass fiber or the like and forms a light guide path for illumination light supplied from the light source device 3.

The lens for illumination 204 is provided at the distal end of the light guide 203. The lens for illumination 204 diffuses light guided by the light guide 203 and emits the light to the outside from the distal end of the endoscope 2. The lens for illumination 204 is configured using one or a plurality of lenses.

The A/D converter 205 A/D-converts analog image data (image signal) generated by the imaging element 201 and outputs converted digital image data to the processor device 4. The A/D converter 205 is configured using an AD conversion circuit configured by a comparator circuit, a reference signal generation circuit, an amplifier circuit, and the like.

The imaging-information storing unit 206 stores data including various programs for operating the endoscope 2, various parameters necessary for the operation of the endoscope 2, and identification information of the endoscope 2. The imaging-information storing unit 206 includes an identification-information storing unit 206a that records the identification information. The identification information includes specific information (ID), a model, specification information, and a transmission scheme of the endoscope 2 and array information of the filters in the color filter 202. The imaging-information storing unit 206 is realized using a flash memory or the like.

The operating unit 207 receives inputs of an instruction signal for switching the operation of the endoscope 2, an instruction signal for causing the light source device to perform a switching operation of illumination light and outputs the received instruction signals to the processor device 4. The operating unit 207 is configured using a switch, a jog dial, a button, a touch panel, and the like.

Configuration of the Light Source Device

A configuration of the light source device 3 is explained. The light source device 3 includes an illuminating unit 31 and an illumination control unit 32.

The illuminating unit 31 supplies illumination lights having wavelength bands different from one another to the light guide 203 under control by the illumination control unit 32. The illuminating unit 31 includes a light source 31a, a light source driver 31b, a switching filter 31c, a driving unit 31d, and a driving driver 31e.

The light source 31a emits illumination light under the control by the illumination control unit 32. The illumination light emitted by the light source 31a is emitted to the outside from the distal end of the endoscope 2 through the switching filter 31c, a condensing lens 31f, and the light guide 203. The light source 31a is realized using a plurality of LED lamps or a plurality of laser light sources that irradiate lights in wavelength bands different from one another. For example, the light source 31a is configured using three LED lamps, that is, an LED 31a_B, an LED 31a_G, and an LED 31a_R.

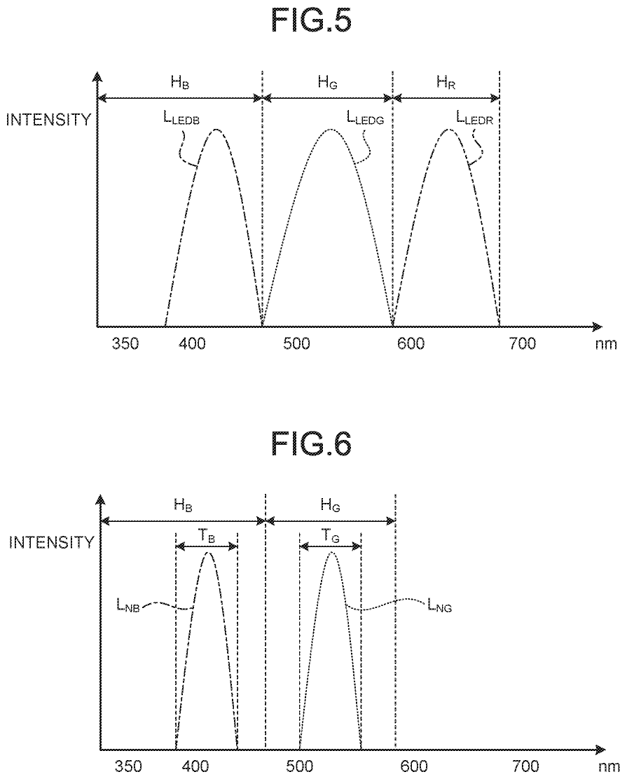

FIG. 5 is a diagram illustrating an example of spectral characteristics of the lights emitted by the light source 31a. In FIG. 5, the horizontal axis indicates a wavelength and the vertical axis indicates intensity. In FIG. 5, a curve L_{LEDB} indicates a spectral characteristic of illumination light of blue irradiated by the LED 31a_B, a curve L_{LEDG} indicates a spectral characteristic of illumination light of green irradiated by the LED 31a_G, and a curve L_{LEDR} indicates a spectral characteristic of illumination light of red irradiated by the LED 31a_R.

As indicated by the curve L_{LEDB} in FIG. 5, the LED 31a_B has peak intensity in the wavelength band H_{B} of blue (for example, 380 nm to 480 nm). As indicated by the curve L_{LEDG} in FIG. 5, the LED 31a_G has peak intensity in the wavelength band H_{G} of green (for example, 480 nm to 580 nm). Further, as indicated by the curve L_{LEDR} in FIG. 5, the LED 31a_R has peak intensity in the wavelength band H_{R} of red (for example, 580 nm to 680 nm).

Referring back to FIG. 1 and FIG. 2, the explanation of the configuration of the endoscope system 1 is continued.

The light source driver 31b supplies an electric current to the light source 31a under the control by the illumination control unit 32 to thereby cause the light source 31a to emit illumination light.

The switching filter 31c is insertably and removably disposed on an optical path of the illumination light emitted by the light source 31a and transmits lights in predetermined wavelength bands in the illumination light emitted by the light source 31a. Specifically, the switching filter 31c transmits narrowband light of blue and narrowband light of green. That is, when the switching filter 31c is disposed on the optical path of the illumination light, the switching filter 31c transmits two narrowband lights. More specifically, the switching filter 31c transmits light in a narrow band T_{B} (for example, 390 nm to 445 nm) included in the wavelength band H_{B} and light in a narrow band T_{G} (for example, 530 nm to 550 nm) included in the wavelength band H_{G}.

FIG. 6 is a diagram illustrating an example of spectral characteristics of the narrowband lights emitted by the light source device 3. In FIG. 6, the horizontal axis indicates a wavelength and the vertical axis indicates intensity. In FIG. 6, a curve LNB indicates a spectral characteristic of the narrowband light in the narrow band T_{B} transmitted through the switching filter 31c and a curve L_{NG} indicates a spectral characteristic of the narrowband light in the narrow band T_{G} transmitted through the switching filter 31c.

As indicated by the curve L_{NB} and the curve L_{NG} in FIG. 6, the switching filter 31c transmits the light in the narrow band T_{B} of blue and the light in the narrow band T_{G} of green. The lights transmitted through the switching filter 31c change to narrowband illumination light including the narrow band T_{B} and the narrow band T_{G}. The narrow bands T_{B} and T_{G} are wavelength bands of blue light and green light easily absorbed by hemoglobin in blood. Observation of an image by the narrowband illumination light is called narrowband light observation scheme (NBI scheme).

Referring back to FIG. 1 and FIG. 2, the explanation of the configuration of the endoscope system 1 is continued.

The driving unit 31d is configured using a stepping motor, a DC motor, or the like and insert the switching filter 31c on the optical path of the illumination light emitted by the light source 31a or retract the switching filter 31c from the optical path under the control by the illumination control unit 32. Specifically, when the endoscope system 1 performs white light imaging (WLI), the driving unit 31d retracts the switching filter 31c from the optical path of the illumination light emitted by the light source 31a under the control by the illumination control unit 32 and, on the other hand, when the endoscope system 1 performs narrow band imaging (NBI), the driving unit 31d inserts (disposes) the switching filter 31c on the optical path of the illumination light emitted by the light source 31a under the control by the illumination control unit 32.

The driving driver 31e supplies a predetermined electric current to the driving unit 31d under the control by the illumination control unit 32.

The condensing lens 31f condenses the illumination light emitted by the light source 31a and emits the illumination light to the light guide 203. The condensing lens 31f condenses the illumination light transmitted through the switching filter 31c and emits the illumination light to the light guide 203. The condensing lens 31f is configured using one or a plurality of lenses.

The illumination control unit 32 is configured using a CPU or the like. The illumination control unit 32 controls the light source driver 31b to turn on and off the light source 31a based on an instruction signal input from the processor device 4. The illumination control unit 32 controls the driving driver 31e to insert the switching filter 31c on and retracts the switching filter 31c from the optical path of the illumination light emitted by the light source 31a based on an instruction signal input from the processor device 4 to thereby control a type (a band) of the illumination light emitted by the illuminating unit 31. Specifically, in the case of sequential lighting, the illumination control unit 32 individually lights at least two LED lamps of the light source 31a and, on the other hand, in the case of simultaneous lighting, the illumination control unit 32 simultaneously lights the at least two LED lamps of the light source 31a to thereby perform control for switching the illumination light emitted from the illuminating unit 31 to one of the sequential lighting and the simultaneous lighting.

Configuration of the Processor Device

A configuration of the processor device 4 is explained.

The processor device 4 performs image processing on image data received from the endoscope 2 and outputs the image data to the display device 5. The processor device 4 includes an image processing unit 41, an input unit 42, a storage unit 43, and a control unit 44.

The image processing unit 41 is configured using a GPU (Graphics Processing Unit), an FPGA (Field Programmable Gate Array), or the like. The image processing unit 41 performs predetermined image processing on the image data and outputs the image data to the display device 5. Specifically, the image processing unit 41 performs OB clamp processing, gain adjustment processing, format conversion processing, and the like besides interpolation processing explained below. The image processing unit 41 includes a detecting unit 411, a generating unit 413, and an interpolating unit 414. Note that, in the first embodiment, the image processing unit 41 functions as an image processing device.

The detecting unit 411 detects positional deviation amounts of pixels among image data of a plurality of frames generated by the imaging element 201. Specifically, the detecting unit 411 detects, using a past image corresponding to image data of a past frame among the plurality of frames and a latest image corresponding to image data of a reference frame (a latest frame), a positional deviation amount (a motion vector) between pixels of the past image and the latest image.

A combining unit 412 combines, based on the positional deviation amounts detected by the detecting unit 411, information concerning pixels in which a first filter is disposed in image data of at least one or more past frames with the image data of the reference frame (the latest frame) to generate combined image data. Specifically, the combining unit 412 combines information (pixel values) concerning G pixels of the past image with information concerning G pixels of the latest image to thereby generate a combined image including half or more G pixels. The combining unit 412 generates a combined image obtained by combining information (pixel values) concerning R pixels of the past image corresponding to the image data of the past frame with information concerning R pixels of the latest image corresponding to the image data of the reference frame (the latest frame) and generates combined image data obtained by combining information (pixel values) concerning B pixels of the past image with information concerning B pixels of the latest image.

The generating unit 413 performs the interpolation processing on the combined image data generated by the combining unit 412 to thereby generate, as reference image data, first interpolated image data including information concerning the first filter in all pixel positions. The generating unit 413 performs, on the combined image generated by the combining unit 412, the interpolation processing for interpolating the information concerning the G pixels to thereby generate, as a reference image, an interpolated image including the information concerning the G pixels in all pixels.

The interpolating unit 414 performs, referring to the reference image data generated by the generating unit 413, the interpolation processing on the image data of the reference frame (the latest frame) to thereby generate, for each of a plurality of types of second filters, second interpolated image data including information concerning the second filter in all pixel positions. Specifically, the interpolating unit 414 performs, based on the reference image generated by the generating unit 413, the interpolation processing on each of the combined image of the R pixels and the combined image of the B pixels generated by the combining unit 412 to thereby generate each of an interpolated image including the information concerning the R pixels in all pixels and an interpolated image including the information concerning the B pixels in all pixels.

The input unit 42 is configured using a switch, a button, a touch panel, and the like, receives an input of an instruction signal for instructing the operation of the endoscope system 1, and outputs the received instruction signal to the control unit 44. Specifically, the input unit 42 receives an input of an instruction signal for switching a scheme of the illumination light irradiated by the light source device 3. For example, when the light source device 3 irradiates the illumination light in the simultaneous lighting, the input unit 42 receives an input of an instruction signal for causing the light source device 3 to irradiate the illumination light in the sequential lighting.

The storage unit 43 is configured using a volatile memory and a nonvolatile memory and stores various kinds of information concerning the endoscope system 1 and programs executed by the endoscope system 1.

The control unit 44 is configured using a CPU (Central Processing Unit). The control unit 44 controls the units configuring the endoscope system 1. For example, the control unit 44 switches, based on the instruction signal for switching the scheme of the illumination light irradiated by the light source device 3 input from the input unit 42, the scheme of the illumination light irradiated by the light source device 3.

Configuration of the Display Device

A configuration of the display device 5 is explained.

The display device 5 receives image data generated by the processor device 4 through a video cable and displays an image corresponding to the image data. The display device 5 displays various kinds of information concerning the endoscope system 1 received from the processor device 4. The display device 5 is configured using a liquid crystal or organic EL (Electro Luminescence) display monitor or the like.

Processing of the Processor Device

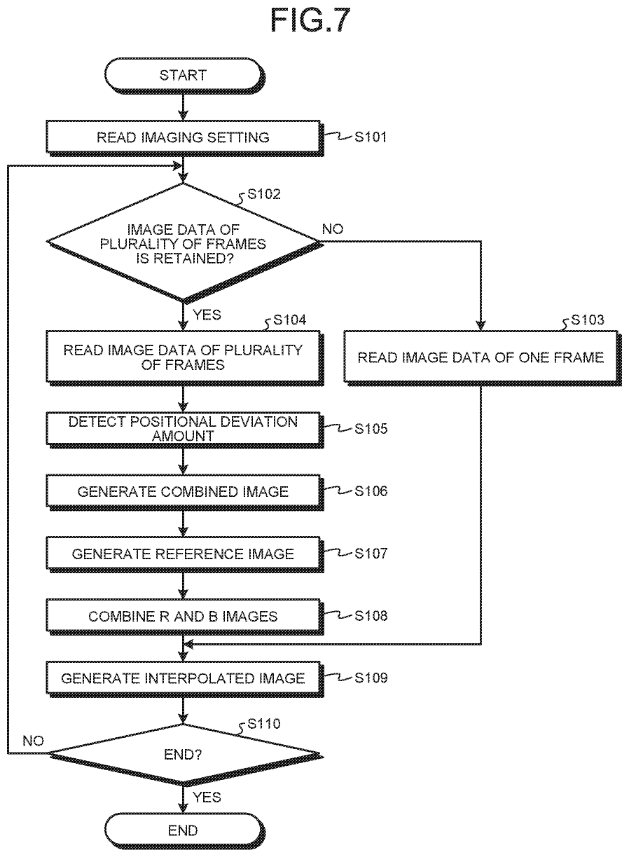

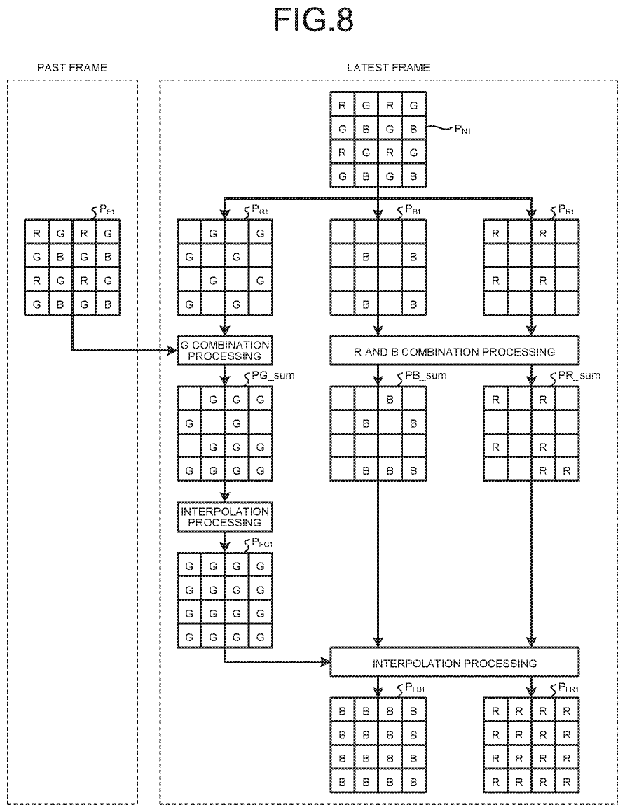

Processing executed by the processor device 4 is explained. FIG. 7 is a flowchart illustrating an overview of the processing executed by the processor device 4. FIG. 8 is a diagram schematically illustrating an image generated by the processor device 4. In FIG. 8, to simplify explanation, image data of one frame (one image) is used as image data of a past frame. However, not only this, but image data of each of a plurality of past frames may be used. Further, in FIG. 7 and FIG. 8, a case where the light source device 3 supplies white light to the endoscope 2 is explained.

As illustrated in FIG. 7, first, when the endoscope 2 is connected to the light source device 3 and the processor device 4 and preparation for starting imaging is made, the control unit 44 reads a driving method for the light source device 3, an observation scheme, and imaging setting for the endoscope from the storage unit 43 and starts capturing of the endoscope 2 (Step S101).

Subsequently, the control unit 44 determines whether image data of a plurality of frames (for example, two or more frames) is retained in the storage unit 43 (Step S102). When the control unit 44 determines that image data of a plurality of frames is retained in the storage unit 43 (Step S102: Yes), the processor device 4 shifts to Step S104 explained below. On the other hand, when the control unit 44 determines that image data of a plurality of frames is not retained in the storage unit 43 (Step S102: No), the processor device 4 shifts to Step S103 explained below.

In Step S103, the image processing unit 41 reads image data of one frame from the storage unit 43. Specifically, the image processing unit 41 reads the latest image data from the storage unit 43. After Step S103, the processor device 4 shifts to Step S109 explained below.

In Step S104, the image processing unit 41 reads image data of a plurality of frames from the storage unit 43. Specifically, the image processing unit 41 reads image data of a past frame and image data of a latest frame from the storage unit 43.

Subsequently, the detecting unit 411 detects a positional deviation amount between the image data of the past frame and the image data of the latest frame (Step S105). Specifically, the detecting unit 411 detects, using a past image corresponding to the image data of the past frame and a latest image corresponding to the image data of the latest frame, a positional deviation amount (a motion vector) between pixels of the past image and the latest image. For example, when alignment processing for two images of the past image and the latest image is performed, the detecting unit 411 detects a positional deviation amount (a motion vector) between the two images and performs alignment with the pixels of the latest image serving as a reference while moving the pixels to eliminate the detected positional deviation amount. As a detection method for detecting a positional deviation amount, existing block matching processing is used. The block matching processing divides an image (a latest image) of a frame (a latest frame) serving as a reference into blocks having fixed size, for example, 8 pixels×8 pixels, calculates, in units of this block, differences from pixels of an image (a past image) of a frame (a past frame) set as a target of the alignment, searches for a block in which a sum (SAD) of the absolute values of the differences is smallest, and detects a positional deviation amount.

Thereafter, the combining unit 412 combines, based on the positional deviation amount detected by the detecting unit 411, information (pixel values) concerning G pixels of a past image corresponding to the image data of the past frame with information concerning G pixels of a latest image corresponding to the image data of the latest frame (Step S106). Specifically, as illustrated in FIG. 8, a latest image P_{N1} includes information concerning half G pixels with respect to the entire image. Accordingly, the combining unit 412 can generate a combined image including the information concerning half or more G pixels by combining the information concerning the G pixels of the past image. For example, as illustrated in FIG. 8, the combining unit 412 combines information (pixel values) concerning G pixels of a past image P_{F1} with information concerning G pixels of a latest image P_{G1} to thereby generate a combined image PG_ₛᵤₘ including information concerning half or more G pixels. Note that, in FIG. 8, to simplify explanation, the past image is only one frame. However, not only this, but the combining unit 412 may combine information concerning G pixels of respective image data of a plurality of past frames with information concerning G pixels of latest frame image data.

Subsequently, the generating unit 413 performs, based on the combined image PG_ₛᵤₘ generated by the combining unit 412, the interpolation processing for interpolating the information concerning the G pixels to thereby generate, as a reference image, an interpolated image including the information concerning the G pixels in all pixels (Step S107). Specifically, as illustrated in FIG. 8, the generating unit 413 performs, on the combined image PG_ₛᵤₘ, the interpolation processing for interpolating the information concerning the G pixels to thereby generate, as a reference image, an interpolated image P_{FG1} including the information concerning the G pixels in all pixels. The G pixels are originally present in half positions with respect to the entire image. Therefore, the G pixels include information in pixels positions compared with the R pixels and the B pixels. Accordingly, the generating unit 413 can generate, as the reference image, the interpolated image P_{FG1} on which the interpolation processing is highly accurately performed by known bilinear interpolation processing, direction discriminating interpolation processing, or the like.

Thereafter, the combining unit 412 combines, based on the positional deviation amount detected by the detecting unit 411, information (pixel values) concerning R pixels of a past image corresponding to image data of a past frame with information concerning R pixels of a latest image P_{R1} corresponding to image data of a latest frame to generate a combined image of the R pixels and combines information (pixel values) concerning B pixels of the past image with information concerning B pixels of the latest image to generate a combined image of the B pixels (Step S108). Specifically, as illustrated in FIG. 8, the combining unit 412 combines information (pixel values) concerning B pixels of the past image P_{F1} with information concerning B pixels of a latest image P_{B1} to generate a combined image PB_ₛᵤₘ of the B pixels and combines information (pixel values) of R pixels of the past image P_{F1} with information concerning R pixels of the latest image P_{R1} to generate a combined image PR_ₛᵤₘ of the R pixels.

Subsequently, the interpolating unit 414 performs, based on the reference image generated by the generating unit 413, the interpolation processing on each of the combined image PR_ₛᵤₘ of the R pixels and the combined image PB_ₛᵤₘ of the B pixels to thereby generate an interpolated image of the R pixels and an interpolated image of the B pixels including the information concerning the R pixels and the B pixels in all pixels of an R image and a B image (Step S109). Specifically, as illustrated in FIG. 8, the interpolating unit 414 performs, based on the reference image (the interpolated image P_{FG}) generated by the generating unit 413, the interpolation processing on each of the combined image PR_ₛᵤₘ and the combined image PB_ₛᵤₘ to thereby generate an interpolated image P_{FR1} including the information concerning the R pixels in all pixels and an interpolated image P_{FB1} including information concerning the B pixels in all pixels. An interpolation method using a reference image is existing joint bilateral interpolation processing, guided filter interpolation processing, or the like. The interpolation processing using a reference image in the past can highly accurately perform interpolation. However, there is a problem in that, when a correlation between information concerning an interpolation target and information concerning the reference image is low, more information concerning the reference image is mixed in an interpolated image as the information concerning the interpolation target is less and color separation performance is deteriorated. On the other hand, according to the first embodiment, before the interpolation processing for the R pixels and the B pixels is performed using the reference image, the combining unit 412 combines the information concerning the respective R pixels and B pixels from the past image to thereby increase information amounts of the R pixels and the B pixels and thereafter the interpolating unit 414 performs the interpolation processing of each of the R pixels and the B pixels. Therefore, the color separation performance can be improved. As a result, a high-resolution image (color image) can be output to the display device 5. Note that, when image data of a past frame is not stored in the storage unit 43, the interpolating unit 414 performs well-known interpolation processing on a latest image corresponding to latest image data to thereby generate images of three colors of the respective R pixels, G pixels, and B pixels and outputs the images to the display device 5.

Thereafter, when receiving an instruction signal for instructing an end from the input unit 42 or the operating unit 207 (Step S110: Yes), the processor device 4 ends this processing. On the other hand, when not receiving the instruction signal for instructing an end from the input unit 42 or the operating unit 207 (Step S110: No), the processor device 4 returns to Step S102 explained above.

According to the first embodiment explained above, the interpolating unit 414 performs, referring to the reference image data generated by the generating unit 413, the interpolation processing on the latest image corresponding to the image data of the latest frame to thereby generate, for each of the plurality of types of second filters, the second interpolated image data including the information concerning the second filter in all the pixel positions. Therefore, even in the simultaneous lighting, it is possible to generate a high-resolution image and output the image to the display device 5.

### Second Embodiment

A second embodiment of the present disclosure is explained. The second embodiment is different from the first embodiment in the configuration of the color filter 202. In the following explanation, a configuration of a color filter in the second embodiment is explained and thereafter processing executed by a processor device according to the second embodiment is explained. Note that the same components as the components of the endoscope system 1 according to the first embodiment explained above are denoted by the same reference numerals and signs and explanation of the components is omitted.

Configuration of the Color Filter

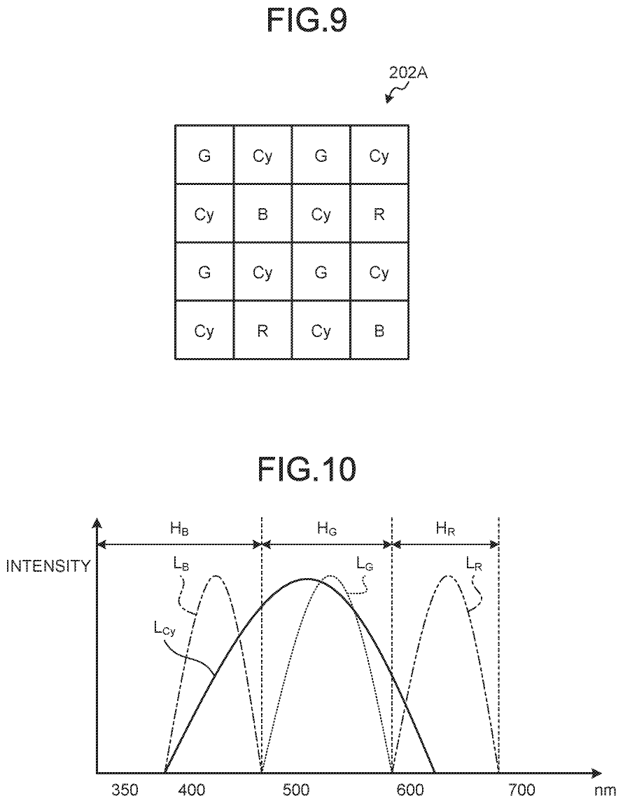

FIG. 9 is a schematic diagram illustrating an example of the configuration of the color filter according to the second embodiment of the present disclosure. A color filter 202A illustrated in FIG. 9 includes sixteen filters arranged in a 4×4 two-dimensional lattice shape. The filters are arranged side by side according to arrangement of pixels. The color filter 202A transmits a wavelength band H_{B} of blue (B), a wavelength band H_{G} of green (G), and a wavelength band H_{R} of red (R). The color filter 202A includes R filters that transmit light in the wavelength band H_{R} of red, G filters that transmit light in the wavelength band H_{G} of green, B filters that transmit light in the wavelength band H_{B} of blue, and Cy filters that transmit the light in the wavelength band of blue and the light in the wavelength band of green. Specifically, in the color filter 202A, the Cy filters are arranged in a checker shape at a ratio (eight) of a half of the entire color filter 202A, the G filters are arranged at a ratio (four) of a quarter of the entire color filter 202A, and each of the filters B and the filters R are arranged at a ratio of one eighth (two).

Transmission Characteristics of the Filters

Transmission characteristics of the filters configuring the color filter 202A are explained. FIG. 10 is a diagram illustrating an example of the transmission characteristics of the filters configuring the color filter 202A. In FIG. 10, transmittance curves are simulatively standardized such that maximum values of transmittances of the filters are equal. In FIG. 10, a curve L_{B} indicates a transmittance curve of the B filter, a curve L_{G} indicates a transmittance curve of the G filter, a curve L_{R} indicates a transmittance curve of the R filter, and a curve L_{Cy} indicates a transmittance curve of the Cy filter. In FIG. 10, the horizontal axis indicates a wavelength and the vertical axis indicates transmittance.

As illustrated in FIG. 10, the Cy filter transmits lights in the wavelength band H_{B} and the wavelength band H_{G} and absorbs (blocks) light in the wavelength band H_{R}. That is, the Cy filter transmits light in a wavelength band of cyan, which is a complementary color. Note that, in this specification, the complementary color means a color formed by lights including at least two wavelength bands among the wavelength bands H_{B}, H_{G}, and H_{R}.

Processing of the Processor Device

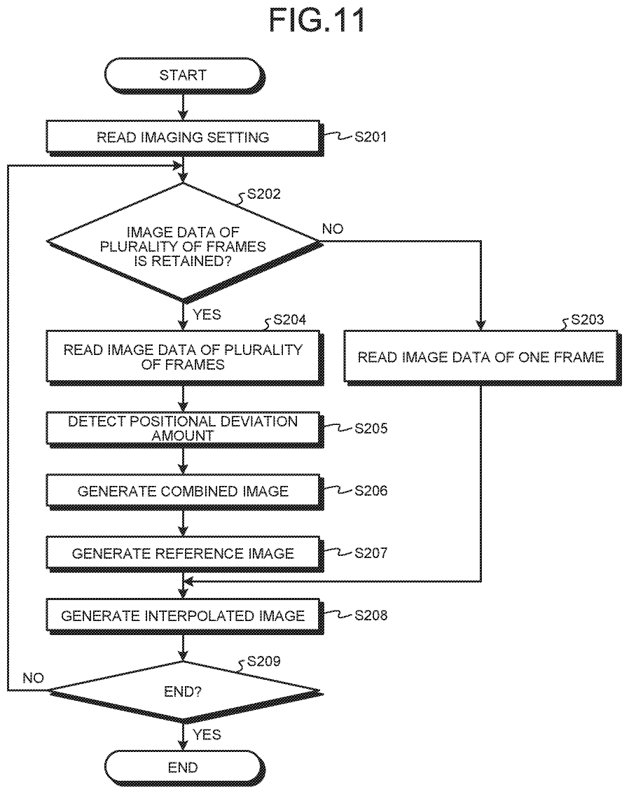

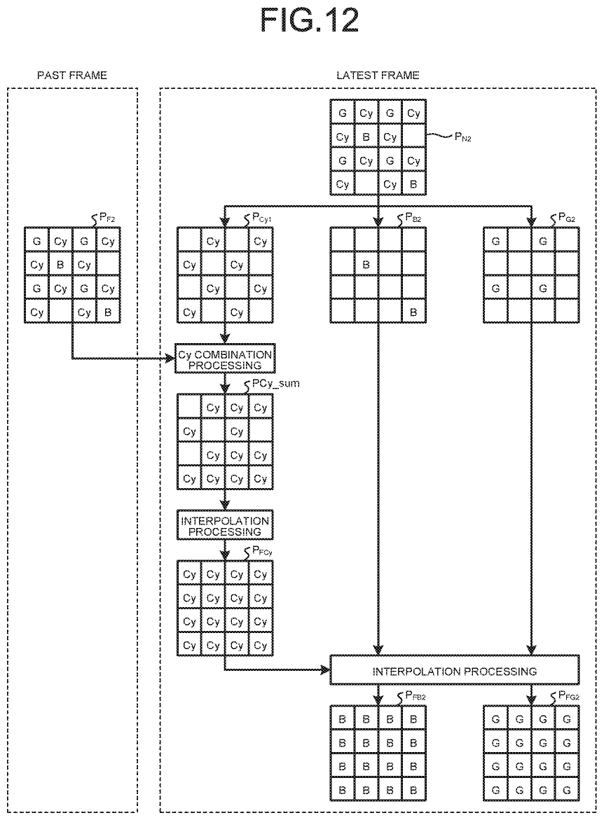

Processing executed by the processor device 4 is explained. FIG. 11 is a flowchart illustrating an overview of the processing executed by the processor device 4. FIG. 12 is a diagram schematically illustrating an image generated by the processor device 4. Note that, in FIG. 12, to simplify explanation, image data of one frame (one image) is used as image data of a past frame. However, not only this, but image data of each of a plurality of past frames may be used. Further, in the following explanation, the light source device 3 supplies narrowband illumination light to the endoscope 2. Note that, when the light source device 3 supplies white light to the endoscope 2, the processor device 4 performs the same processing as the processing in the first embodiment to generate respective R, G, and B images.

In FIG. 11, Step S201 to S205 correspond to respective Step S101 to S105 in FIG. 7 explained above.

In Step S206, the combining unit 412 combines, based on the positional deviation amount detected by the detecting unit 411, information (pixel values) concerning Cy pixels of a past image P_{F2} corresponding to the image data of the past frame with information concerning Cy pixels of a latest image P_{Cy1} corresponding to the image data of the latest frame. A latest image P_{F1} includes information concerning half Cy pixels with respect to the entire image. Accordingly, as illustrated in FIG. 12, the combining unit 412 can generate a combined image PCy_ₛᵤₘ including information concerning half or more Cy pixels by combining the information concerning the Cy pixels of the past image P_{F2} with the latest image P_{Cy1}. Note that, in FIG. 12, to simplify explanation, a past image is only one frame. However, not only this, but the combining unit 412 may combine information concerning Cy pixels of image data of each of a plurality of past frames with information concerning Cy pixels of latest frame image data.

Subsequently, the generating unit 413 performs, based on the combined image generated by the interpolating unit 414, the interpolation processing for interpolating the information concerning the Cy pixels to thereby generate, as a reference image, an interpolated image including the information concerning the Cy pixels in all pixels (Step S207). Specifically, as illustrated in FIG. 12, the generating unit 413 performs, on the combined image PCy_ₛᵤₘ, the interpolation processing for interpolating the information concerning the Cy pixels to thereby generate, as the reference image, an interpolated image P_{FCy} including the information concerning the Cy pixels in all pixels of an image. The Cy pixels are originally present in half positions with respect to all the pixels. Therefore, the Cy pixels include information in pixel positions compared with the G pixels and the B pixels. Accordingly, the generating unit 413 can generate, as the reference image, an interpolated image P_{FCy} on which the interpolation processing is highly accurately performed by known bilinear interpolation processing, direction discriminating interpolation processing, or the like.

Subsequently, the interpolating unit 414 performs, based on the reference image generated by the generating unit 413, the interpolation processing on each of the combined image of the B pixels and the combined image of the G pixels to thereby generate an interpolated image of the B pixels and an interpolated image of the G pixels including the information concerning the B pixels and the G pixels in all pixels of the B image and the G image (Step S208). Specifically, as illustrated in FIG. 12, the interpolating unit 414 performs the interpolation processing using information (an image P_{B2}) of the B pixels and information (an image P_{G2}) of the G pixels included in the reference image (the interpolated image P_{FCy}) generated by the generating unit 413 and the latest image P_{N2} to thereby generate an interpolated image P_{FB2} of the B pixels and an interpolated image P_{FG2} of the G pixels. The Cy pixels arranged in the checker shape have a high correlation with the B pixels and the G pixels. Accordingly, even when information amounts (pixel values) of the B pixels and the G pixels are small, the interpolating unit 414 can highly accurately perform the interpolation processing while keeping color separation performance by performing the interpolation processing using at least the reference image (the interpolated image P_{FCy}) of the Cy pixels. Consequently, when the endoscope 2 performs the narrow band imaging, the endoscope system 1 can output a high-resolution image. After Step S208, the processor device 4 shifts to Step S209. Step S209 corresponds to Step S109 in FIG. 7 explained above.

According to the second embodiment explained above, even when information amounts (pixel values) of the B pixels and the G pixels are small, the interpolating unit 414 can highly accurately perform the interpolation processing while keeping the color separation performance by performing the interpolation processing of each of the B pixels and the G pixels using the reference image (the interpolated image P_{FCy}) of the Cy pixels. Therefore, it is possible to improve the color separation performance. Moreover, it is possible to save combination processing for the B pixels and the G pixels.

### Third Embodiment

A third embodiment of the present disclosure is explained below. The third embodiment is different from the second embodiment in a configuration of an image processing unit 41. Specifically, in the third embodiment, it is determined based on a positional deviation amount whether an interpolated image using a reference image is generated. In the following explanation, a configuration of an image processing unit according to the third embodiment is explained and thereafter processing executed by a processor device according to the third embodiment is explained.

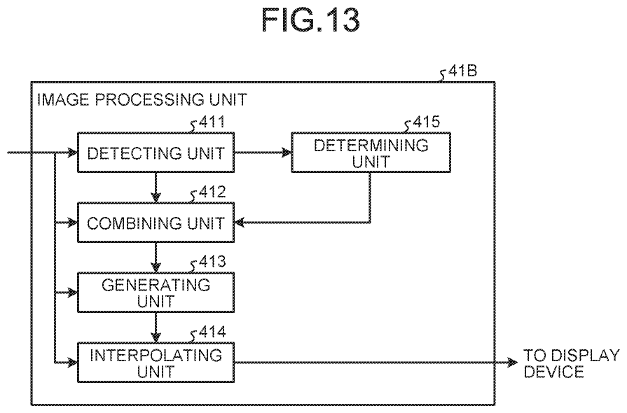

FIG. 13 is a block diagram illustrating a functional configuration of the image processing unit according to the third embodiment of the present disclosure. An image processing unit 41B illustrated in FIG. 13 further includes a determining unit 415 in addition to the components of the image processing unit 41 according to the second embodiment.

The determining unit 415 determines whether a positional deviation amount detected by the detecting unit 411 is smaller than a threshold.

Processing of the Processor Device

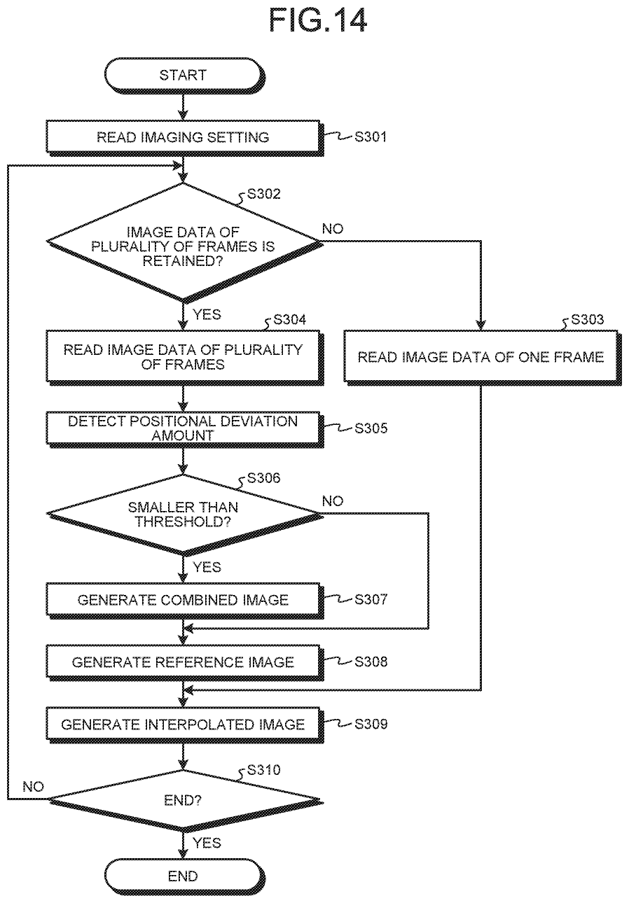

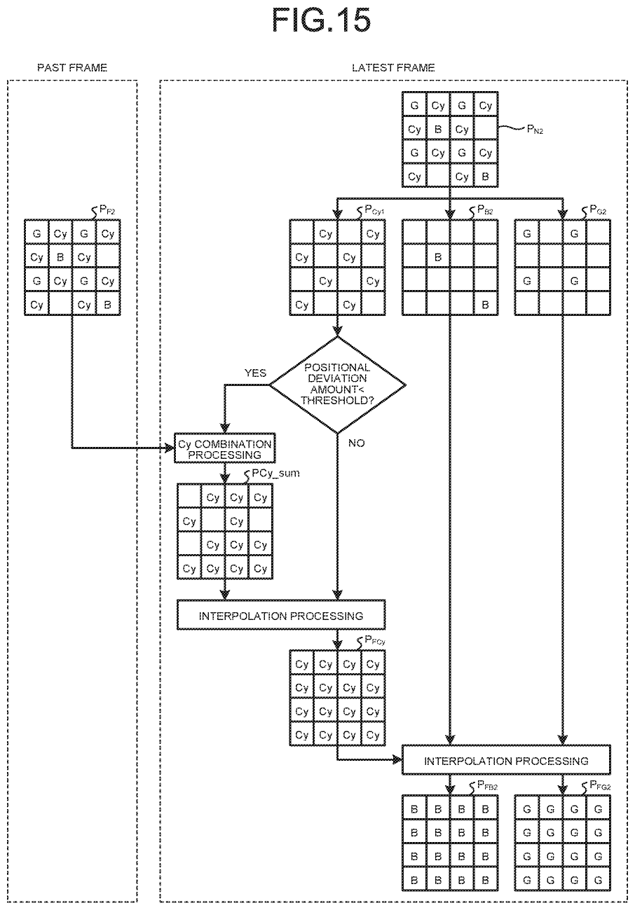

Processing executed by the processor device 4 is explained. FIG. 14 is a flowchart illustrating an overview of the processing executed by the processor device 4. FIG. 15 is a diagram schematically illustrating an image generated by the processor device 4. Note that, in FIG. 15, to simplify explanation, image data of one frame (one image) is used as image data of a past frame. However, not only this but image data of each of a plurality of past frames may be used. Further, in the following explanation, the light source device 3 supplies narrowband illumination light to the endoscope 2. Note that, when the light source device 3 supplies white light to the endoscope 2, the processor device 4 performs the same processing as the processing in the first embodiment to generate respective R, G, and B images.

In FIG. 14, Step S301 to S305 respectively correspond to Step S101 to S105 in FIG. 7 explained above.

In Step S306, the determining unit 415 determines whether the positional deviation amount detected by the detecting unit 411 is smaller than a threshold. When the determining unit 415 determines that the positional deviation amount detected by the detecting unit 411 is smaller than the threshold (Step S306: Yes), the processor device 4 shifts to Step S307 explained below. On the other hand, when the determining unit 415 determines that the positional deviation amount detected by the detecting unit 411 is not smaller than the threshold (Step S306: No), the processor device 4 shifts to Step S308 explained below.

In Step S307, the combining unit 412 combines, based on the positional deviation amount detected by the detecting unit 411, information (pixel values) concerning Cy pixels of a past image P_{F2} corresponding to image data of a past frame with information concerning Cy pixels of a latest image P_{Cy1} corresponding to image data of a latest frame. Specifically, as illustrated in FIG. 15, the combining unit 412 combines the information concerning the Cy pixels of the past image P_{F2} with the latest image P_{Cy1} to thereby generate a combined image PCy_ₛᵤₘ including information concerning half or more Cy pixels. After Step S307, the processor device 4 shifts to Step S308 explained below. Note that, in FIG. 15, to simplify explanation, a past image is only one frame. However, not only this, but the combining unit 412 may combine information concerning Cy pixels of image data of each of a plurality of past frames with information concerning Cy pixels of latest frame image data.

Subsequently, the generating unit 413 performs, based on the combined image generated by the interpolating unit 414 or the latest image, the interpolation processing for interpolating the information concerning the Cy pixels to thereby generate, as a reference image, an interpolated image including the information concerning the Cy pixels in all pixels of an image (Step S308). Specifically, when the determining unit 415 determines that the positional deviation amount detected by the detecting unit 411 is smaller than the threshold and the combining unit 412 generates a combined image, the generating unit 413 performs, on the combined image Cy_ₛᵤₘ, the interpolation processing for interpolating the information concerning the Cy pixels to thereby generate, as the reference image, an interpolated image P_{FCy} including the information concerning the Cy pixels in all pixels of an image. On the other hand, when the determining unit 415 determines that the positional deviation amount detected by the detecting unit 411 is not smaller than the threshold, the generating unit 413 performs, on information (a latest image P_{Cy1}) concerning Cy pixels of a latest image P_{N2}, the interpolation processing for interpolating the information concerning the Cy pixels to thereby generate, as the reference image, an interpolated image P_{FCy} including the information concerning the Cy pixels in all pixels. That is, in the case of a scene in which a movement amount (a positional deviation amount) during screening or the like of a lesion of a subject by the endoscope 2 is large, since resolution is relatively not important, the generating unit 413 generates a reference image using image data of only one frame.

Step S309 and Step S310 respectively correspond to Step S208 and Step S209 in FIG. 11 explained above.

According to the third embodiment explained above, when the determining unit 415 determines that a positional deviation amount detected by the detecting unit 411 is smaller than the threshold and the combining unit 412 generates a combined image, the generating unit 413 performs the interpolation processing for interpolating the information concerning the Cy pixels with respect to the combined image Cy_ₛᵤₘ to thereby generates, as the reference image, the interpolated image P_{FCy} including the information concerning the Cy pixels in all pixels of an image. Therefore, in addition to the effects in the second embodiment explained above, it is possible to generate an optimum reference image according to a movement amount of a scene. Even in a scene in which a movement is large, it is possible to generate an output image without causing artifact.

### Fourth Embodiment

A fourth embodiment of the present disclosure is explained. In the second embodiment explained above, the information concerning the Cy pixels of the past image and the information concerning the Cy pixels of the latest image are simply combined based on the positional deviation amount. However, in the fourth embodiment, weighting in combining the information is performed based on the positional deviation amount and the information is combined. In the following explanation, processing executed by a processor device according to the fourth embodiment is explained. Note that the same components as the components of the endoscope system 1 according to the second embodiment explained above are denoted by the same reference numerals and signs and detailed explanation of the components is omitted.

Processing of the Processor Device

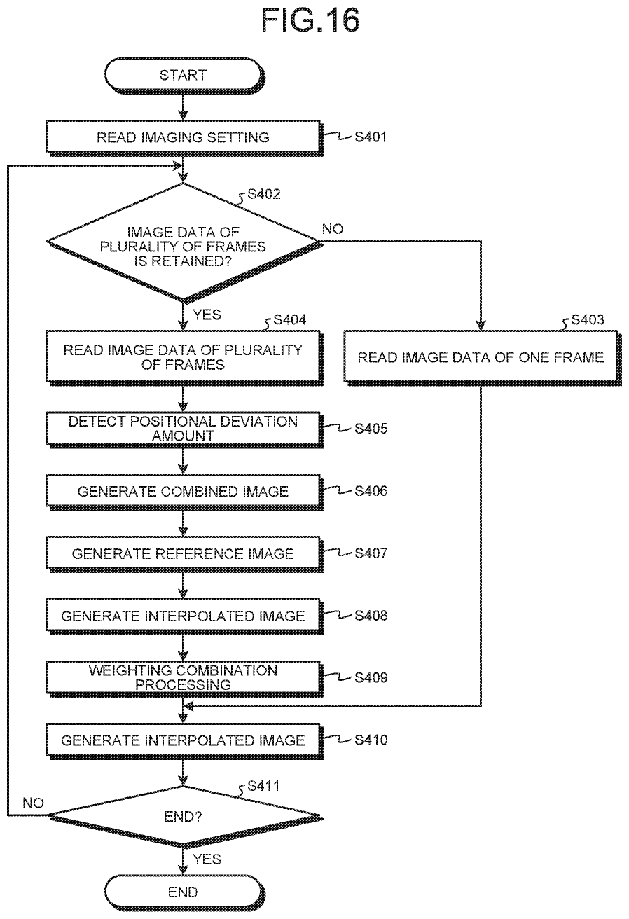

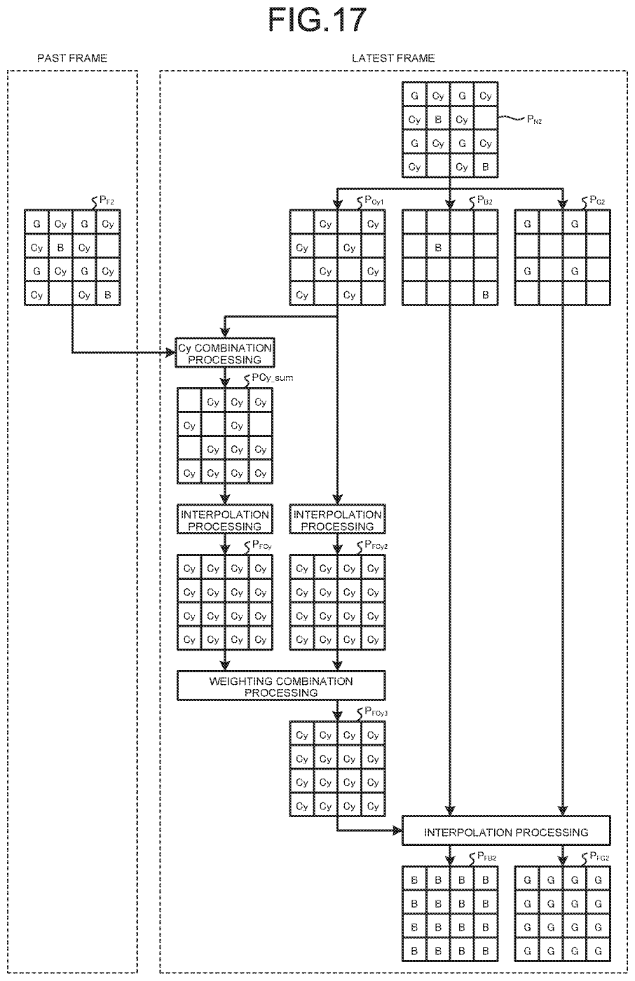

FIG. 16 is a flowchart illustrating an overview of the processing executed by the processor device. FIG. 17 is a diagram schematically illustrating an image generated by the processor device 4. In FIG. 17, to simplify explanation, image data of one frame (one image) is used as image data of a past frame. However, not only this, but image data of each of a plurality of past frames may be used. Further, in the following explanation, the light source device 3 supplies narrowband illumination light to the endoscope 2. Note that, when the light source device 3 supplies white light to the endoscope 2, the processor device 4 performs the same processing as the processing in the first embodiment to generate respective R, G, and B images.

In FIG. 16, Step S401 to S407 respectively correspond to Step S101 to S107 in FIG. 7 explained above.

In Step S408, the generating unit 413 performs interpolation processing on Cy pixels of a latest image corresponding to image data of a latest frame to thereby generate an interpolated image including information concerning the Cy pixels in all pixels. Specifically, as illustrated in FIG. 17, the generating unit 413 performs the interpolation processing on a latest image P_{Cy1} of the Cy pixels to thereby generate an interpolated image P_{FCy2} including the information concerning the Cy pixels in all pixels.

Subsequently, the generating unit 413 generates, based on a positional deviation amount detected by the detecting unit 411, new reference image data combined by performing weighting of reference image data generated using combined image data and reference image data generated using image data of a latest frame (a reference frame) (Step S409). Specifically, as illustrated in FIG. 17, when the positional deviation amount detected by the detecting unit 411 is smaller than a threshold, the generating unit 413 performs weighting such that a ratio of the reference image F_{Cy} is higher with respect to the reference image F_{Cy2} and generates a reference image P_{FCy3}. For example, when the positional deviation amount detected by the detecting unit 411 is smaller than the threshold, the generating unit 413 combines the reference image F_{Cy2} and the reference image F_{Cy} through weighting at a combination ratio of 9:1 to thereby generate the reference image P_{FCy3}. On the other hand, when the positional deviation amount detected by the detecting unit 411 is not smaller than the threshold, the generating unit 413 performs weighting such that the ratio of the reference image F_{Cy} is small with respect to the reference image F_{Cy2} and generates the reference image P_{FCy3}.

Step S410 and Step S411 respectively correspond to Step S109 and Step S110 in FIG. 7.

According to the fourth embodiment explained above, the generating unit 413 generates, based on the positional deviation amount detected by the detecting unit 411, new reference image data combined by performing weighting of reference image data generated using combined image data and reference image data generated using image data of a latest frame (a reference frame). Therefore, it is possible to reduce a sudden image quality change during switching of use of image data of a plurality of frames and use of image data of only one frame.

### Other Embodiments

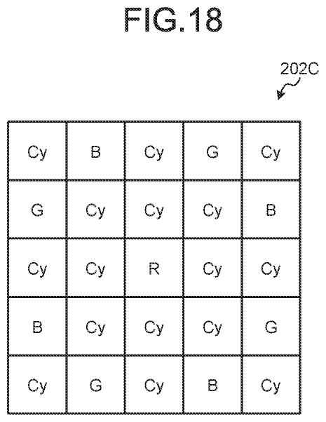

In the first to fourth embodiments explained above, the configuration of the color filter can be changed as appropriate. FIG. 18 is a schematic diagram illustrating an example of a configuration of a color filter according to a modification of the first to fourth embodiments of the present disclosure. As illustrated in FIG. 18, a color filter 202C includes twenty-five filters arranged in a 5×5 two-dimensional lattice shape. In the color filter 202C, Cy filters are arranged at a ratio (sixteen) of a half or more of the entire color filter 202C, four G filters are arranged, four B filters are arranged, and two R filters are arranged.

Various embodiments can be formed by combining, as appropriate, a plurality of components disclosed in the first to fourth embodiments of the present disclosure. For example, several component may be deleted from all the components described in the first to fourth embodiments of the present disclosure explained above. Further, the components explained in the first to fourth embodiments of the present disclosure explained above may be combined as appropriate.

In the first to fourth embodiments of the present disclosure, the processor device and the light source device are separate. However, the processor device and the light source device may be integrally formed.

The first to fourth embodiments of the present disclosure are applied to the endoscope system. However, the first to fourth embodiments can also be applied to, for example, an endoscope of a capsule type, a video microscope that images a subject, a cellular phone having an imaging function and an irradiating function of irradiating illumination light, and a tablet terminal having an imaging function.

The first to fourth embodiments of the present disclosure are applied to the endoscope system including the flexible endoscope. However, the first to fourth embodiments can also be applied to an endoscope system including a rigid endoscope and an endoscope system including an industrial endoscope.

The first to fourth embodiments of the present disclosure are applied to the endoscope system including the endoscope inserted into a subject. However, the first to fourth embodiments can also be applied to, for example, an endoscope system including a rigid endoscope and an endoscope system such as a paranasal sinus endoscope, an electric knife, and a test probe.

In the first to fourth embodiments of the present disclosure, “unit” described above can read “means”, “circuit”, and the like. For example, the control unit can read control means and a control circuit.

A program to be executed by the endoscope system according to the first to fourth embodiments of the present disclosure is provided while being recorded in a computer-readable recording medium such as a CD-ROM, a flexible disk (FD), a CD-R, a DVD (Digital Versatile Disk), a USB medium, or a flash memory as file data of an installable form or an executable form.

The program to be executed by the endoscope system according to the first to fourth embodiments of the present disclosure may be provided by being stored on a computer connected to a network such as the Internet and downloaded through the network. Further, the program to be executed by the endoscope system according to the first to fourth embodiments of the present disclosure may be provided or distributed through a network such as the Internet.

In the first to fourth embodiments of the present disclosure, data is bidirectionally transmitted and received via a cable. However, not only this, but the processor device may transmit, on the network, a file storing image data generated by the endoscope through a server or the like.

In the first to fourth embodiments of the present disclosure, a signal is transmitted from the endoscope to the processor device via a transmission cable. However, for example, the signal does not need to be transmitted by wire and may be wirelessly transmitted. In this case, an image signal and the like only have to be transmitted from the endoscope to the processor device according to a predetermined wireless communication standard (for example, Wi-Fi (registered trademark) or Bluetooth (registered trademark)). Naturally, the wireless communication may be performed according to other wireless communication standards.

Note that, in the explanation of the flowcharts in this specification, an anteroposterior relation of the processing among the steps is clearly indicated using expressions such as “first”, “thereafter”, and “subsequently”. However, the order of the processing necessary for carrying out the present disclosure is not uniquely decided by the expressions. That is, the order of the processing in the flowcharts described in this specification can be changed in a range without contradiction.

According to the present disclosure, there is an effect that it is possible to generate a high-resolution image even with image data captured by an imaging element having filter arrangement in which primary color filters and complementary color filters are mixed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An image processing device comprising a processor comprising hardware, the image processing device to which an endoscope is connectable, the endoscope including an image sensor and a color filter, the image sensor including a plurality of pixels arranged in a two-dimensional lattice shape, each pixel being configured to receive and photoelectrically convert lights to generate image data in a predetermined frame, the color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the pixels,
the processor being configured to:
detect a positional deviation amount of the pixels among the image data of a plurality of frames generated by the image sensor;
combine, based on the detected positional deviation amount, information concerning the pixels, in which the first filter is arranged, of the image data of at least one or more past frames with image data of a reference frame to generate combined image data;
perform interpolation processing on the generated combined image data to generate, as reference image data, first interpolated image data including information concerning the first filter in all pixel positions; and
perform, referring to the generated reference image data, interpolation processing on the image data of the reference frame to generate, for each of the plurality of types of second filters, second interpolated image data including information concerning the second filters in all pixel positions.

2. The image processing device according to claim 1, wherein the processor is further configured to
determine whether the detected positional deviation amount is smaller than a threshold,
when it is determined that the detected positional deviation amount is smaller than the threshold, generate the reference image data using the combined image data, and
when it is determined that the detected positional deviation amount is not smaller than the threshold, perform interpolation processing on the image data of the reference frame to generate the reference image data.

3. The image processing device according to claim 2, wherein the processor is configured to generate, based on the detected positional deviation amount, a new version of the reference image data combined by performing weighting of the reference image data generated using the combined image data and the reference image data generated using the image data of the reference frame.

4. An endoscope system comprising:
an endoscope configured to be inserted into a subject; and
an image processing device to which the endoscope is connected, wherein
the endoscope includes:
an image sensor in which a plurality of pixels arranged in a two-dimensional lattice shape, each pixel being configured to receive and photoelectrically convert lights to generate image data in a predetermined frame; and
a color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the pixels, and
the image processing device includes a processor comprising hardware, the processor being configured to:
detect a positional deviation amount of the pixels among the image data of a plurality of frames generated by the image sensor;
combine, based on the detected positional deviation amount, information concerning the pixels, in which the first filter is arranged, of the image data of at least one or more past frames with image data of a reference frame to generate combined image data;
perform interpolation processing on the generated combined image data to generate, as reference image data, first interpolated image data including information concerning the first filter in all pixel positions; and
perform, referring to the generated reference image data, interpolation processing on the image data of the reference frame to generate, for each of the plurality of types of second filters, second interpolated image data including information concerning the second filters in all pixel positions.

5. An image processing method executed by an image processing device to which an endoscope is connectable, the endoscope including an image sensor and a color filter, the image sensor including a plurality of pixels arranged in a two-dimensional lattice shape, each pixel being configured to receive and photoelectrically convert lights to generate image data in a predetermined frame, the color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the pixels, the image processing method comprising:
detecting a positional deviation amount of the pixels among the image data of a plurality of frames generated by the image sensor;
combining, based on the detected positional deviation amount, information concerning the pixels, in which the first filter is arranged, of the image data of at least one or more past frames with image data of a reference frame to generate combined image data;
performing interpolation processing on the generated combined image data to generate, as reference image data, first interpolated image data including information concerning the first filter in all pixel positions; and
performing, referring to the generated reference image data, interpolation processing on the image data of the reference frame to generate, for each of the plurality of types of second filters, second interpolated image data including information concerning the second filters in all pixel positions.

6. A non-transitory computer-readable recording medium with an executable program stored thereon, the program causing an image processing device to which an endoscope is connectable, the endoscope including image sensor and a color filter, the image sensor including a plurality of pixels arranged in a two-dimensional lattice shape, each pixel being configured to receive and photoelectrically convert lights to generate image data in a predetermined frame, the color filter including a first filter and a plurality of types of second filters, the first filter being arranged in half or more pixels of all the pixels in the image sensor and being a cyan filter configured to transmit light in a wavelength band of blue and light in a wavelength band of green, the second filters having spectral sensitivity characteristics different from a spectral sensitivity characteristic of the first filter, the first filter and the second filters being arranged to correspond to the plurality of pixels, to execute:
detecting a positional deviation amount of the pixels among the image data of a plurality of frames generated by the image sensor;
combining, based on the detected positional deviation amount, information concerning the pixels, in which the first filter is arranged, of the image data of at least one or more past frames with image data of a reference frame to generate combined image data;
performing interpolation processing on the generated combined image data to generate, as reference image data, first interpolated image data including information concerning the first filter in all pixel positions; and
performing, referring to the generated reference image data, interpolation processing on the image data of the reference frame to generate, for each of the plurality of types of second filters, second interpolated image data including information concerning the second filters in all pixel positions.
